(19) United States
(12) Patent Application Publication
(10) Pub. No.: US 2004/0193070 A1
(43) Pub. Date: Sep. 30, 2004

(54) Systems and methods for detecting and mapping disorders of the macular portion of the retina

(76) Inventors: Meyer Schilder, Jenkintown, PA (US); Paul J. Missischia, Havertown, PA (US)

Correspondence Address: ANTHONY L. CUPOLI, 111 BUNNING DRIVE, VOORHEES, NJ, 08043, US

(21) Appl. No.: 10/401,357

(22) Filed: Mar. 28, 2003

(51) Int. Cl. A61B 13/00

(52) U.S. Cl. 600/558

(57) ABSTRACT

A method is provided for detecting vision loss due to disorders of the macular portion of the eye. A person being tested is positioned to view a color video display monitor on which the test is displayed at a distance suitable for testing the macular portion of the eye. The person responds to targets presented and the response is recorded for printing to show the area of the eye in which there is vision loss.

The invention relates to a proprietary computer software program and related apparatus for detecting and mapping disorders of the macular portion of the retina.

### BACKGROUND

Today, the foremost destroyer of vision among older individuals is Age Related Macular Degeneration (ARMD). It is now the leading cause of blindness in older adults. This age-related disease is particularly sinister because it occurs without warning. The onset of the disease is painless and the affected individual does not know that he/she has it until the macular vision is lost and the individual's ability to read and recognize detail is lost.

It is estimated that ARMD affects over 8 million adults, and with the aging of our population, many more will suffer from ARMD. Although the cause of ARMD is not known, within the last five years, the scientific community has been and is now, devoting millions of dollars in search of a treatment and hopefully, a cure for this disease. It is suspected that heredity plays a part, but why some are afflicted and others are not is not known. Nor is there an explanation for why ARMD can affect one eye and not the other.

The onset of the disease is first recognized when the individual suddenly realizes that what he/she sees is not normal (a straight line has a dip in it, straight lines become curved, etc.). A visit to a primary care physician refers the individual to an ophthalmologist, who in turn refers the patient to a retinal specialist for treatment. If caught at a very early stage (“wet” macular degeneration”), the progression of the disease can be slowed or even arrested (a treatment using Visudyne™ has proven very successful). However once the vision has been lost, it cannot be retrieved.

At the present time only two diagnostic aides are available to the eye-care professional. They are (1) the Amsler Grid and (2) Fluorescein with ICG angiography.

The Amsler Grid is a rectangular grid printed on paper. For the test, the patient is shown the preprinted grid containing parallel horizontal and parallel vertical lines, which intersect at a 90-degree angle. If the patient indicates that he/she cannot see the entire grid he/she is instructed to encircle the “cannot see” area to indicate where the macular “hole” is. This test is used by ophthalmologists to diagnose individuals with ARMD. The test (1) requires the presence of a technician or other medical personnel during the test, (2) produces an anecdotal result, and (3) produces poorly defined results because older individuals are more likely to have hand-to-eye coordination limitations. Moreover, the results cannot be saved and later retrieved from an auxiliary storage medium such as a magnetically recorded computer disk.

Retinal specialists image the actual, physical condition of the macula using Fluorescein in ICG angiography. In this procedure, the patient is injected in the arm with Fluorescein dye and a fundus camera is used to photograph the dye passing through the retina. Although this procedure produces an accurate picture of the current physical condition of the macula, the technique can only be performed in a hospital or other controlled environment where appropriate medical equipment is available in case of a adverse reaction on the part of the patient to the injection. This procedure is invasive and the cost is not inconsequential. Although the result defines the physical aspects of the patient's retina, it does not necessarily indicate what the patient can actually see because the cognitive value of the human brain often compensates for physical defect and can increase the ability of an individual (with ARMD) to recognize small objects.

Diagnosis of ARMD is made by an eye-care professional such as an ophthalmologist. Currently, the absence of any noninvasive diagnostic tool other than the Amsler Grid for testing a patient indicates a need for an economical non-invasive diagnostic device for detecting the onset and progression of ARMD. Ophthalmologists have sometimes utilized a device called the visual field analyzer (one such device is made by Humphrey Instruments Corp., a spin-off from SKF Industries). This device is primarily used for determining visual field defects from various ocular diseases, but does not provide sufficient detail of the macular area to be useful in imaging macular degeneration problems or other macular disorders.

### SUMMARY

The solution is a simple, economical, non-invasive diagnostic tool that can be used by eye-care professionals such as ophthalmologists and optometrists, and may ultimately be used by primary care physicians (the general practitioner who sees the patient regularly), and perhaps others. The invention described in this patent disclosure is such a diagnostic tool. The tool includes a computer, a video display unit, an keyboard-input device, and a specialized program for communication between the tool and the test taker and the test evaluator.

This is a method for detecting vision loss due to disorders of the macular portion of the eye comprising the steps:

a) providing a color video display monitor having a display of three concentric areas, an innermost area corresponding to macular vision, an immediately surrounding second concentric area corresponding to paramacular vision and an outer third concentric area corresponding to extra-macular vision; a fixation point at the center of the concentric areas and cross-hairs as an aid in finding the fixation point;

b) positioning a person at distance from the display suitable for his macular, paramacular and extra-macular vision to correspond to the three concentric test areas,

c) selecting the macular area for testing the patient;

d) recording to a patient demographic file the identity of the person and the eye selected for testing;

e) painting a target in the desired concentric test area in a manner that appears random to the person for a period of time suitable for testing for the ability of the person to see that target,

f) providing a response mechanism to the person to indicate that he is seeing the target, wherein the response mechanism is suitably responsive to permit a timely response;

g) detecting the response of the person to the target;

h) recording to the patient demographic file any lack of response;

i) repeating steps “e” through “g” by redisplaying the target randomly both temporally and spatially in the desired test area until the complete area has been tested, and then

j) recalling the patient demographic file containing the targets not seen, the identity of the person and the eye tested and presenting it on the display or other medium.

The software program is also a subject of this invention. The software package produces a digital image based on the patient's response to a stimulus presented on a video display screen. Information from a “Patient Profile” database identifies the test results, and the results are recorded in a “Test Results” database.

The digital picture of the test results can readily be recalled, printed on paper or re-displayed on the video display screen at any time. The software is programmed in the commonly used “C” programming language but will be available in any of the commonly used programming languages.

### DESCRIPTION OF THE PREFERRED EMBODIMENTS

In order to generate a digital map of a portion of an individual's retina, a target (such as a filled-in circle or square) must be shown to the individual being tested and whether-or-not the individual “sees” the target must be saved. The inability of the tested individual to “see” the target may indicate the presence of the disease known as Macular Degeneration (assuming neither surgery nor injury to the eye has caused the condition). The totality of targets not seen by the individual comprises the “macular defect” in the individual's vision. Using a computer with a video display screen for displaying the target and a patient response device such as a key on a keyboard or a button that can be pressed, the individual being tested signals the computer that he/she “sees” the displayed target. To correlate the test results with the individual's eye being tested, pertinent information such as the test ID (test number, date and time) and a patient number (identifying the patient being tested), can be and preferably is be fed to the computer program. Before starting the test, in a preferred practice, the individual initiating the test enters the patient number and “right eye or left eye” information as an input to the program and the tester visually verifies these “test controls”.

For the test, the individual is positioned at a specified distance from the display screen and instructed to fix the focus of the eye being tested on the fixation point at the center of the display screen. To assist the individual, a chin and headrest 14 can be provided. During the test, the eye not being tested is covered or blocked so that only the eye being tested can views the display screen. At the specified distance the individual's eye can view three (concentric) areas. The central area is viewed by the macula of the eye. The area immediately surrounding the central area is designated the paramacular area. The remaining area is referred to as the extra-macular area. The macular area is used for reading and recognizing small symbols such as letters, numbers, etc. The paramacular can be used for seeing small figures if the macular has been damaged. The extra-macular area provides the individual's peripheral vision. To accurately generate a digital map of any area, the computer must display the target in every location in the area being tested. The target remains on the screen for a few seconds, sufficient for the individual being tested to respond. After that the target disappears and then after a random number of seconds reappears at another location and the individual is expected to respond to the new target's appearance. The choice of location appears random to the individual being tested.

Color plays a significant role in the process of seeing, and the best color for one individual may not be the same for another. For that reason, the background screen colors and foreground (target) color are preferably capable of being varied. In addition, because older individuals need more time to respond, the time between target presentations is preferably adjustable. A properly sized target is required to properly test the entire area being viewed, and different size targets are required for different tests. A screening test requires a smaller target than a full test of the macula. Target size for a paramacular-screening test may allow a larger size target. The size of the target determines the number or locations that must be illuminated to cover the entire area being tested.

Completion of the test occurs when the target has been displayed in all locations in the area being tested. In order to re-display the test results, the locations “not seen” are saved together with the individual's (patient) number and the test control data (number, date, time, etc.). A hard-copy printout of the test provides a digital map of the portion of the eye that was tested.

The Test Program software monitors the interaction of the patient being tested and the display being shown. A standard 15″ video display monitor (CRT, LCD, plasma, or other display) is preferred at a 14 inch viewing distance. Displays of varying sizes can be used. The display can be attached to a microprocessor with random access memory, graphics capability, and I/O interfaces capable of employing the components specified herein; or alternatively an IBM compatible computer, or other computer. The program is preferably written in a portable language, such as “C.” However, it can be written in any language that can use machine language commands.

The test consists of a random display of a graphic such as a filled-in circle or other geometric shape. The target appears at a random location on the display screen, disappears, and then reappears at another location on the screen. The target appears randomly within the area of the screen that is viewed by the patient when positioned at an appropriate distance from the display screen.

The patient is instructed to press a button (or some other response device) to indicate that he/she sees the target. To ensure that the patient does not anticipate the appearance of a target, the elapsed time between appearances of the target is varied. In order to properly generate the digital image, the eye of the patient being tested must be positioned and remain at a specified distance from the display. To insure that the patient is focusing directly on the center of the display screen, on which the target is being displayed, the patient's chin can be positioned on a fixed chin rest 13 and the patient's head can be pressed against a fixed headrest 18. Such a fixating device is standard equipment in an eye-care professional's examination room (the apparatus that holds the slit-lamp device used by the eye-care professional for checking the patient's eye). If the test is performed in an environment other than an eye-care professional's examination room, a similar fixturing device is supplied with the system.

The display on the video display monitor is divided into three concentric areas. The central (macular) area viewed from a distance of 14″ is represented by a 2″ square, the surrounding (paramacular) area is represented by a concentric 5″ square, and the remaining (extra-macular) area is represented by an 8″ concentric circle. When macular degeneration occurs, the loss of central vision is the result. When macular degeneration occurs, the brain will attempt to use the paramacular area “to see”. The system can test either the macular or the paramacular+macular areas. Upon completion of the test (all locations in either the macular or paramacular area) having been tested, the program presents a display showing all the targets that were not signaled by the patient as having been seen. This test result is saved in the “Test Results” database, identified by the patient number and test number (patient number having been assigned by the “Patient Profile” database software and the test number being assigned sequentially by the Test program.

The screen is divided into the three areas—macular, paramacular and extra-macular. At a distance of 14″, the macula can view the central 2″ area and the paramacular can view the central 5″ of the screen. A circle reaching from the top of the screen to the bottom of the screen represents the extramacular area. A fixation point at the center and a pair of cross hairs reaching from the edge of the screen to the outline of the paramacular area assist the patient in maintaining his fixation on the center of the screen. In this way, eye movement (which would nullify the results of the test) is prevented.

The basic test for macular disorder, including macular degeneration, involves displaying a ¼″ target in each of the 64 locations, which comprises the screen area that is viewed by the macula at the nominal distance of 14″. If any defects are detected, a much finer scan (full test mode) is used in which a ⅛″ target is displayed in each of the 256 locations that make up the 2″ macular viewing area. A paramacular-screening test is also provided in which a ⅜″ target is displayed in each of the 169 locations, which make up the area viewed by the paramacular area. In each case, the use of random placement and varied time between target displays prevents the patient from anticipating the next target appearance, which would affect the reliability of the test.

For purposes of verifying that the correct patient profile can be recorded with the test results, a “patient/test” identification box is preferably displayed at the top left corner of the test screen. In addition, when a test result is recalled, the test result image, which appears, also displays test controls (number, date, time and right or left eye) as well as the data derived from the patient profile (pupil size, visual acuity and corrective lens prescription).

A default set of parameters can be is pre-programmed. These parameters include:

1. Background colors for: macular, paramacular, extra-macular areas.

2. Target color.

3. Test area (macular or paramacular).

4. Screening Test or full test.

5. Target movement rate (one of three speeds).

The Main Menu, which appears at startup of the program, provides for altering the pre-programmed (default) parameters. A simple keystroke selects one of the Main Menu options. An audible sound can be used to provide useful information. For example, three “beeps” can be sounded when the test is completed, and a single “beep” can be used to alert the technician or doctor to non-normal conditions.

In addition to sending the images and test results to the auxiliary storage (database), the program uploads the patient profile from the auxiliary storage (database). The patient profile must be entered prior to running the test.

### BRIEF DESCRIPTION OF THE DRAWINGS

FIG. 1 is a view of a chin and head rest

FIG. 2 is a schematic overview of a test system

FIG. 3 is a is a diagrammatic representation of a test screen

FIG. 4 is a diagrammatic representation of a test in progress test screen

FIG. 5 is a diagrammatic representation of the test results screen

FIG. 5A is a side view of a person taking the test.

FIG. 5B is a side view of an eye viewing a test display through a cone.

FIG. 5C is a view of the fixation point of the display see through the cone.

FIG. 6 is an alternative diagrammatic representation of the test results screen.

FIG. 7 is a diagrammatic representation of the main test menu

FIG. 7A is a flow chart for the testing program

FIG. 7B is a patient selection flow chart

FIG. 7C is a run test flow chart.

FIG. 7D is a flow chart for changing background colors and target controls

FIG. 7E is a flow chart for re-displaying or printing test results

FIG. 8 is a self-explanatory set of drawing symbol keys.

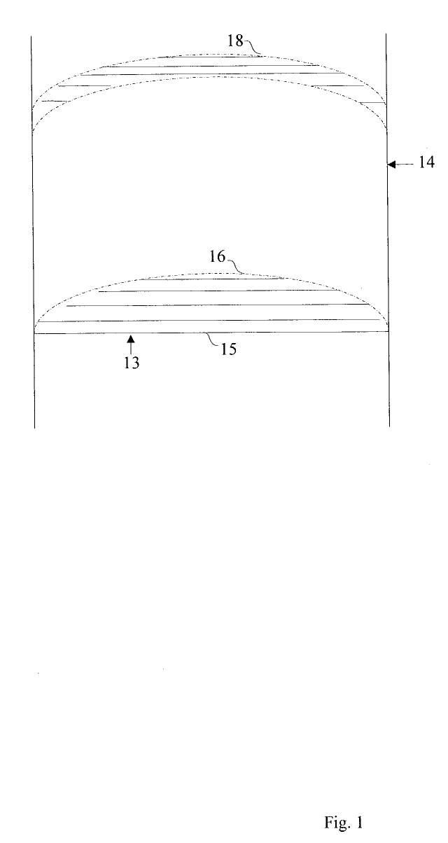

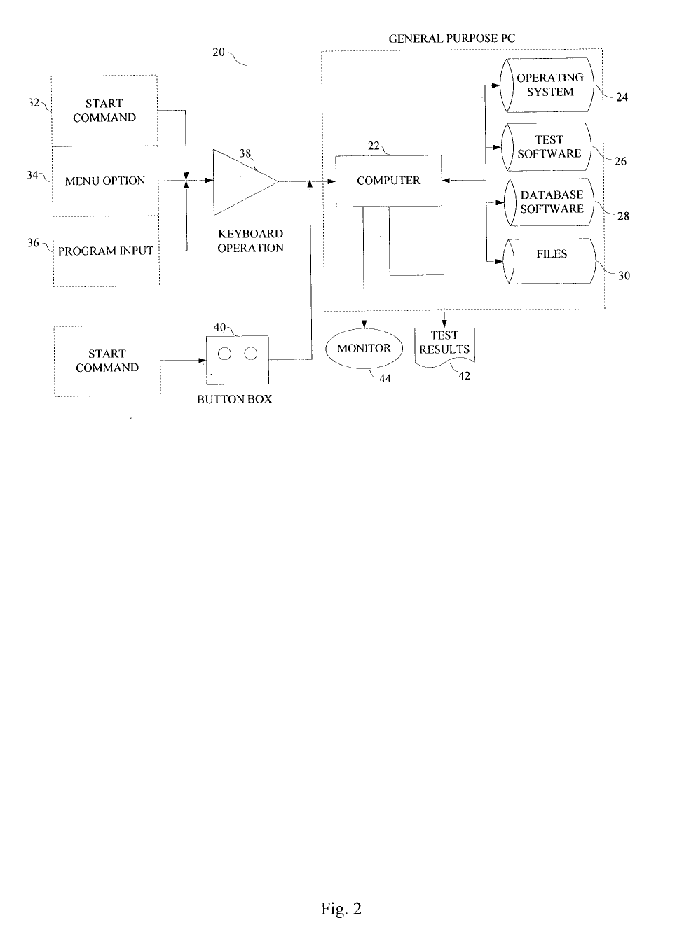

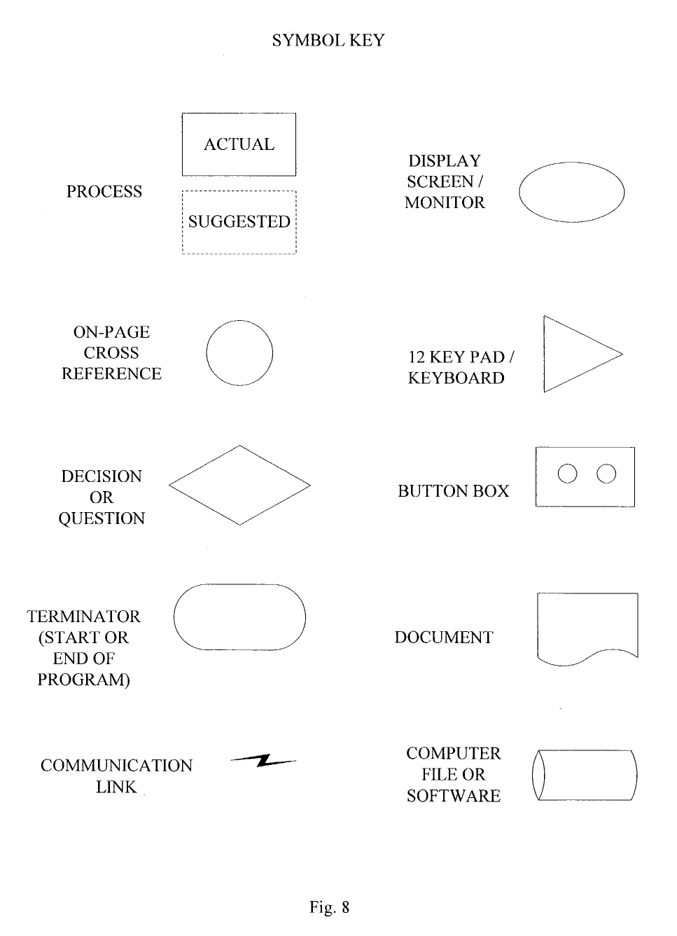

A simple depiction of a test system 20 is shown in FIG. 2. A computer 22 having an operating system 24, test software 26, database software 28, and patient test result files 30 is shown. The start command 32, menu options 34 and program input 36 can be accessed from a keyboard 38 by either the test giver or the test taker. Alternatively, the test can be started and taken using a button box 40, which preferably has one or two buttons. The button box depicted has two buttons: one pausing the computer and the other for all other input activities. Test results 42 can be viewed on a monitor 44 or can otherwise be output. Head support 14 shown in FIG. 1 provides for head stabilization and aids in centering the eye being tested on the screen. Head support 14 includes a chin platform 13 and forehead support 18. Chin platform 13 has a forward projecting edge 16 that project towards a video screen and a rear edge 15 that is closest to the person's body. Head support 18 is a band that the forehead of the test taker rests against.

Computer 22 can be an off the shelf computer or it can be a customized computer. Operating system 24 can be any standard operating system. A stable operating system such as Linux or DOS is preferred. Test software 26 can be built from C, Linux, or other customizable software using the flow sheets and information provided herein.

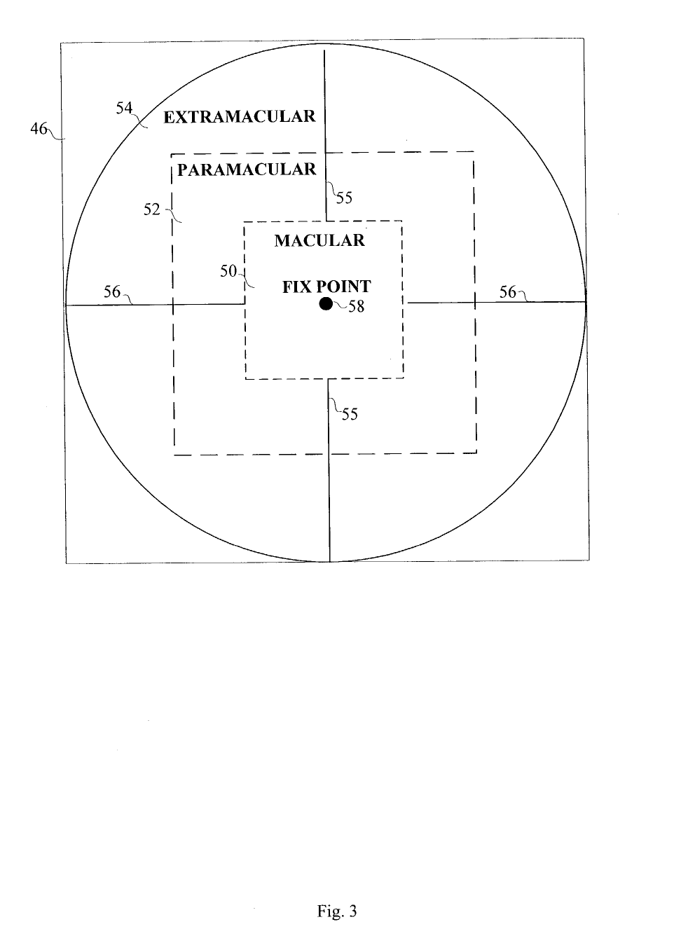

Typically, the test taker is positioned in front of monitor 44. The initial test target screen 46 is shown in FIG. 3. The target screen 46 shows a series of concentric areas. Macular area 50 is the innermost, the most central, area. Para macular area 52 surrounds and borders the Macular 50 area. Extra-macular area 54 is the outermost area depicted. It borders and surrounds Para macular area 52. Vertical lines 55 and horizontal lines 56 appear help the viewer. And in fact, do aid in focusing the viewer's attention on the center 58 of screen 46.

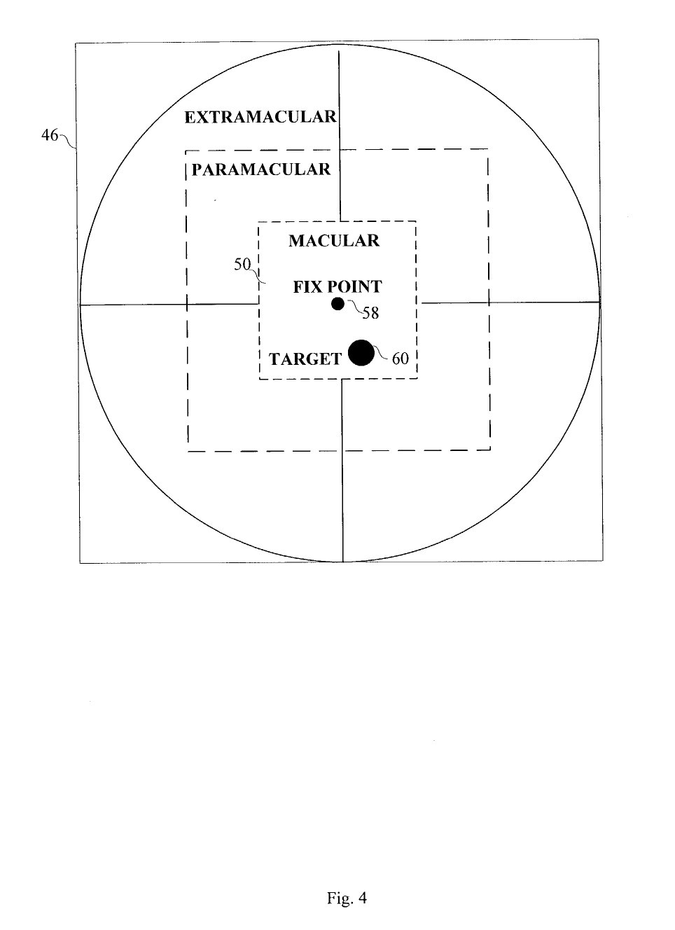

In FIG. 4, macular testing is in progress. The test taker views macular area 50 in FIG. 3, and focusing on the center of the test screen, fix point 58 is presented with target 60. After a few seconds target 60 disappears, and after some random period of time a new target appears for a short random period of time. Each new target is generated in a manner that appears to be random to the viewer, both temporally and spatially. Testing is complete when all the locations that can be viewed have had a target displayed thereon for testing. The program directs the computer to present the target in this random manner.

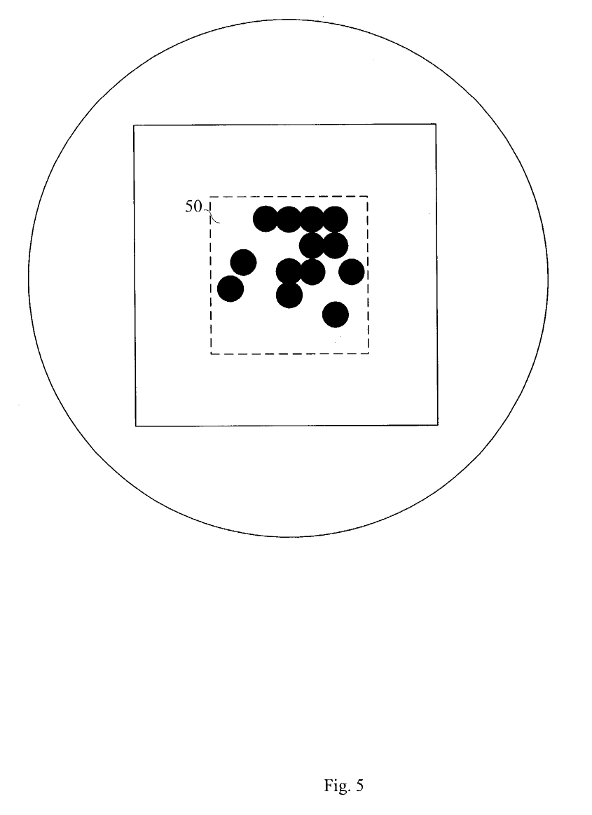

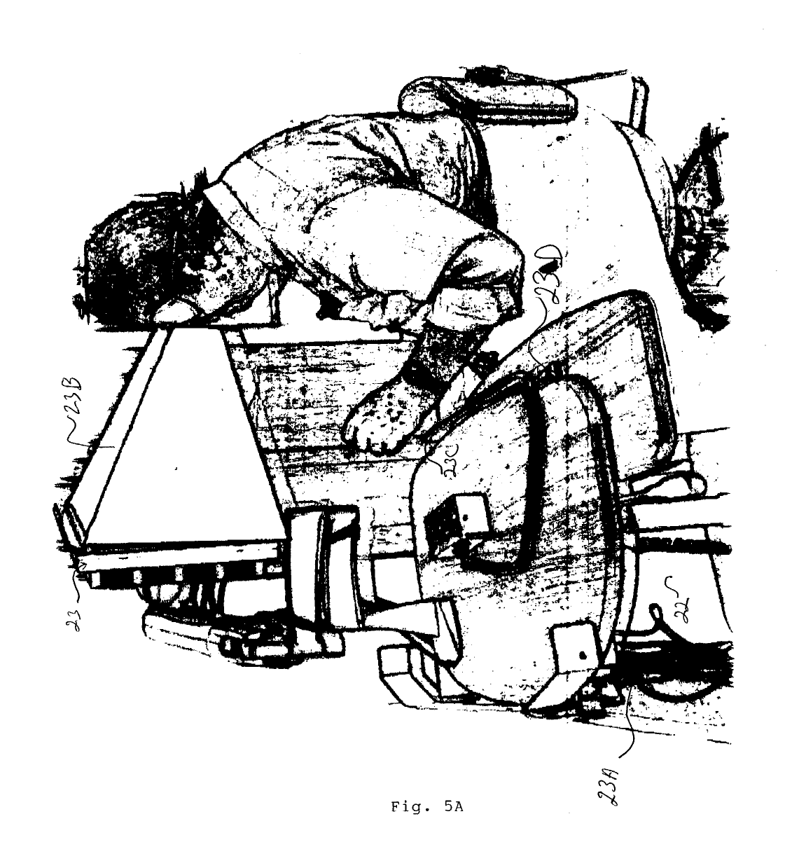

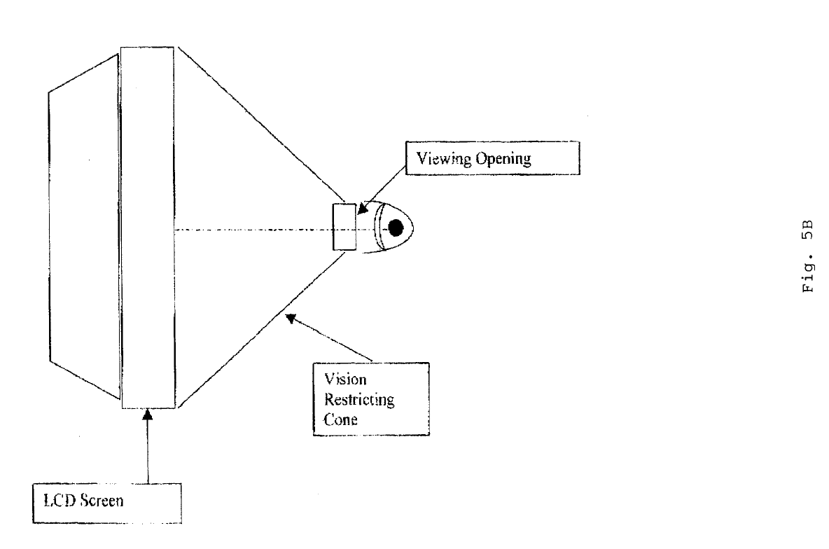

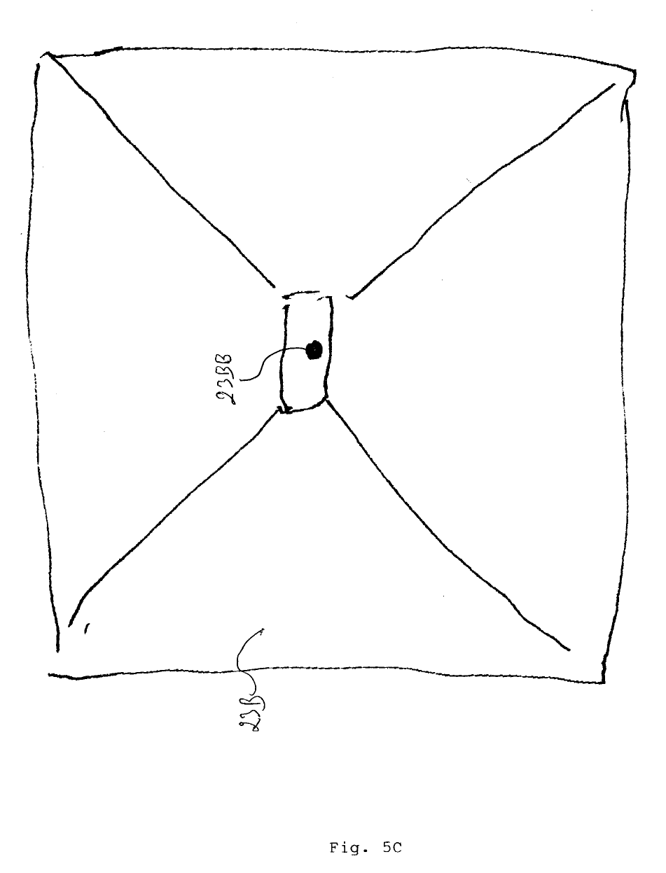

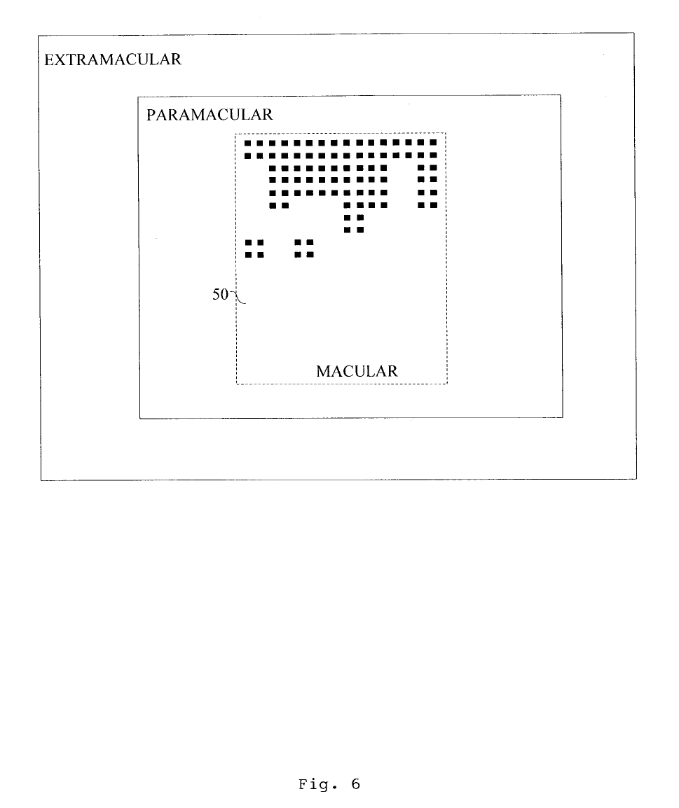

Test results are shown for the finished test in FIG. 5 and in FIG. 6. FIG. 5 also includes information on the test subject, the test number, the date of the test, the eye that was tested, the patient number, the pupil size, the vision, and prescription. This information is useful in identifying and counseling the test subject. FIG. 5 and 6 depict alternative test result views and different target sizes. Larger target sizes are typically used for screening tests and smaller target sizes are used the complete diagnostic test.

FIG. 5A, 5B, and 5C show test equipment in use. A computer 22 is connected to a video display device 23 using wires 23A. Video display device 23 has a conical hood 23B that serves as a positioning device for the person taking the test. Hood 23B has an opening that is aligned generally with the fixation point 23BB on the video display and through which a person views the display while taking the test. Response device 23C held in the right hand of the person is used to indicate that a target has been seen on the video display. A corner of Keyboard 23D is seen on the shelf below the main table platform.

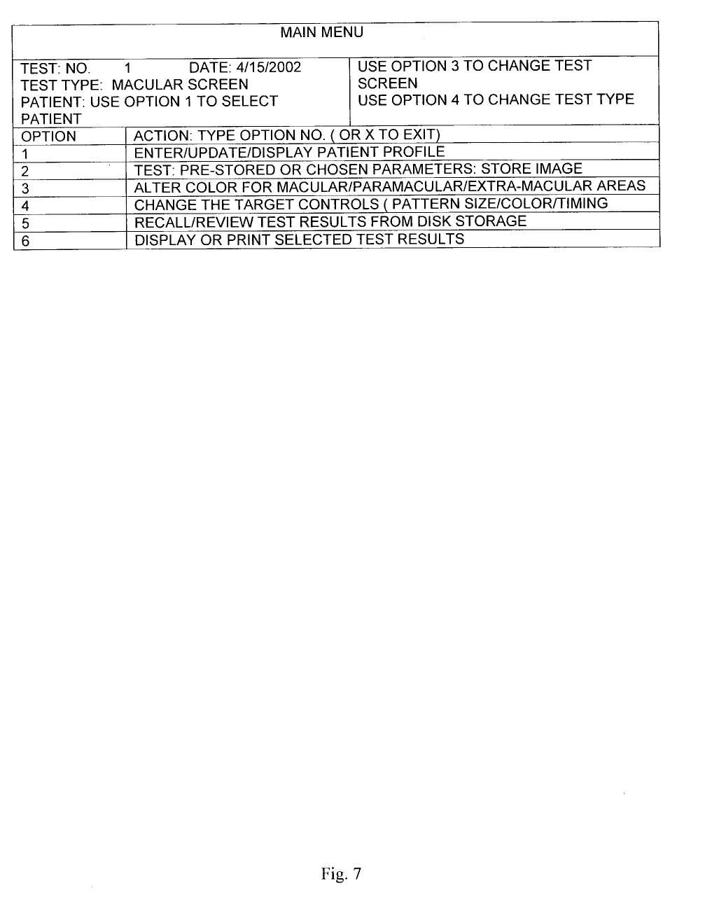

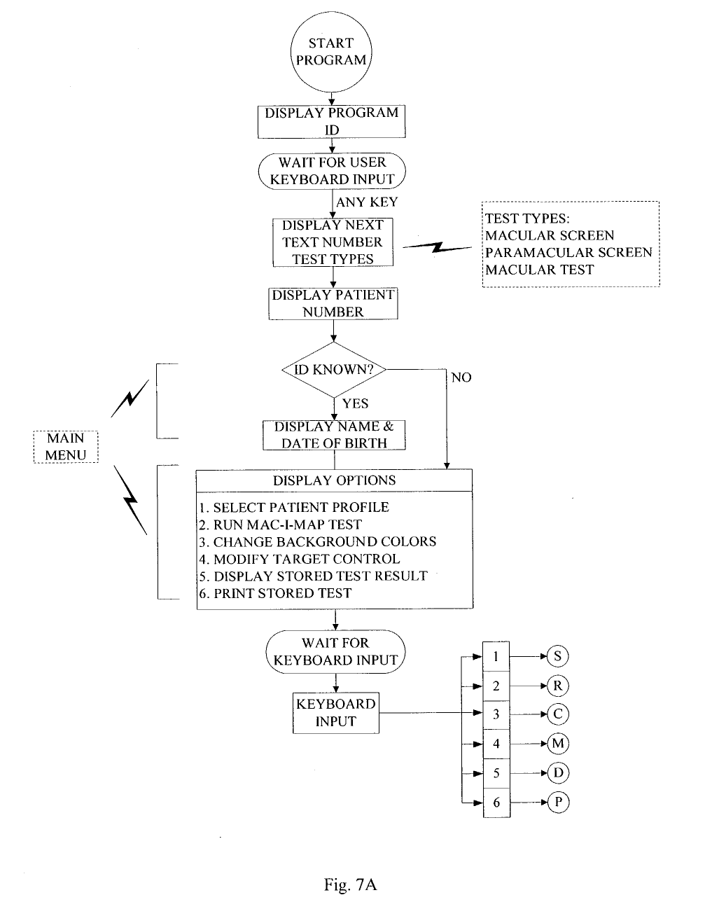

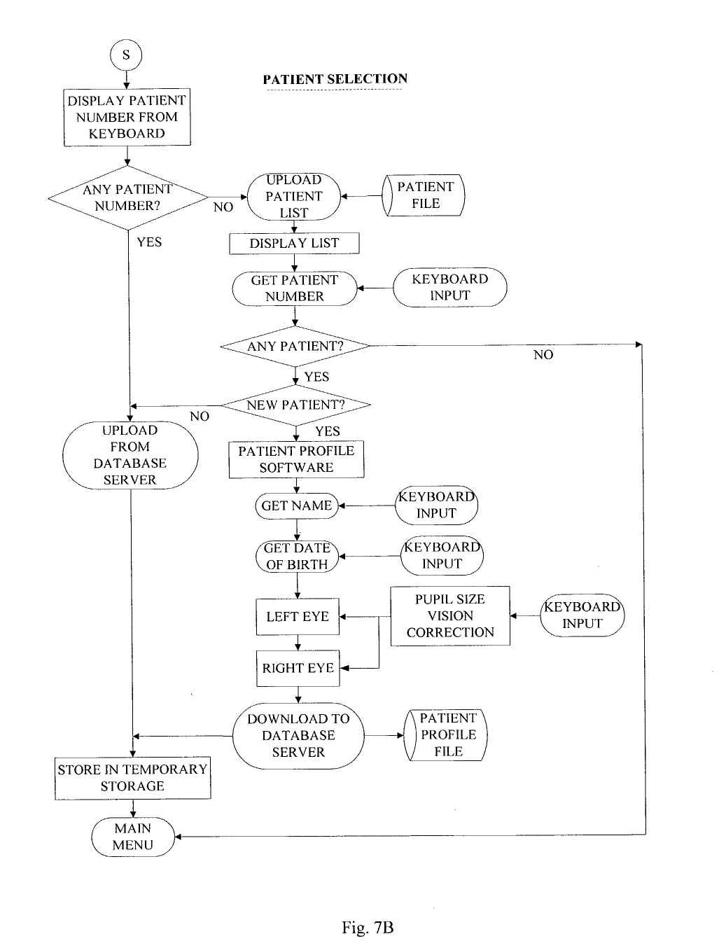

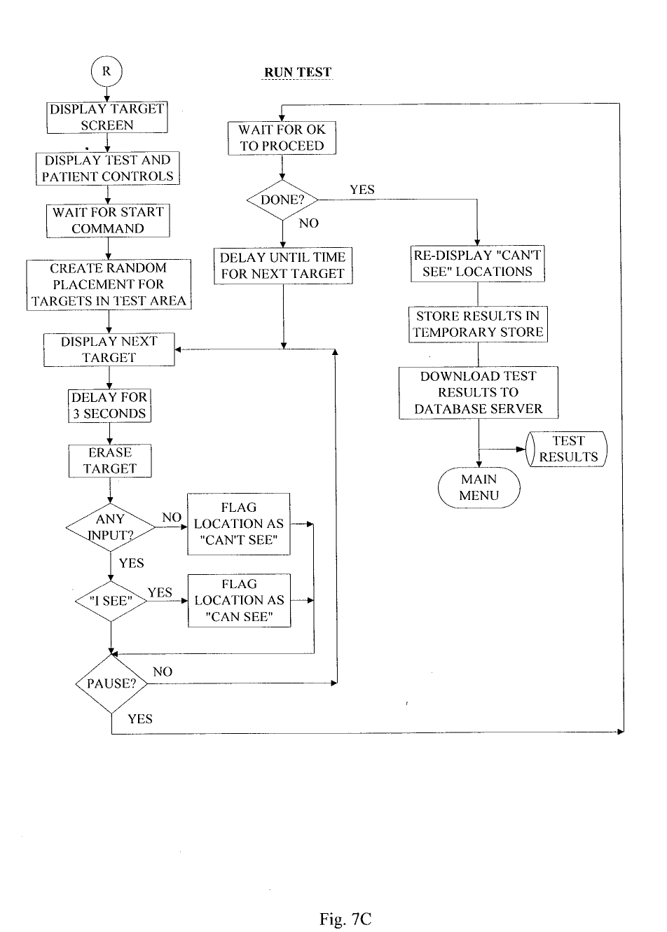

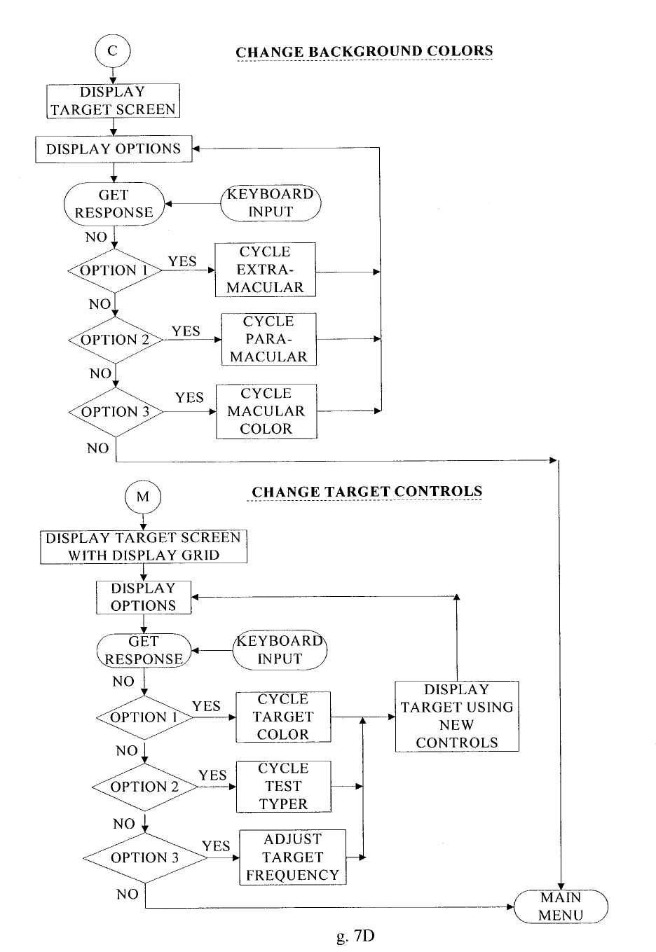

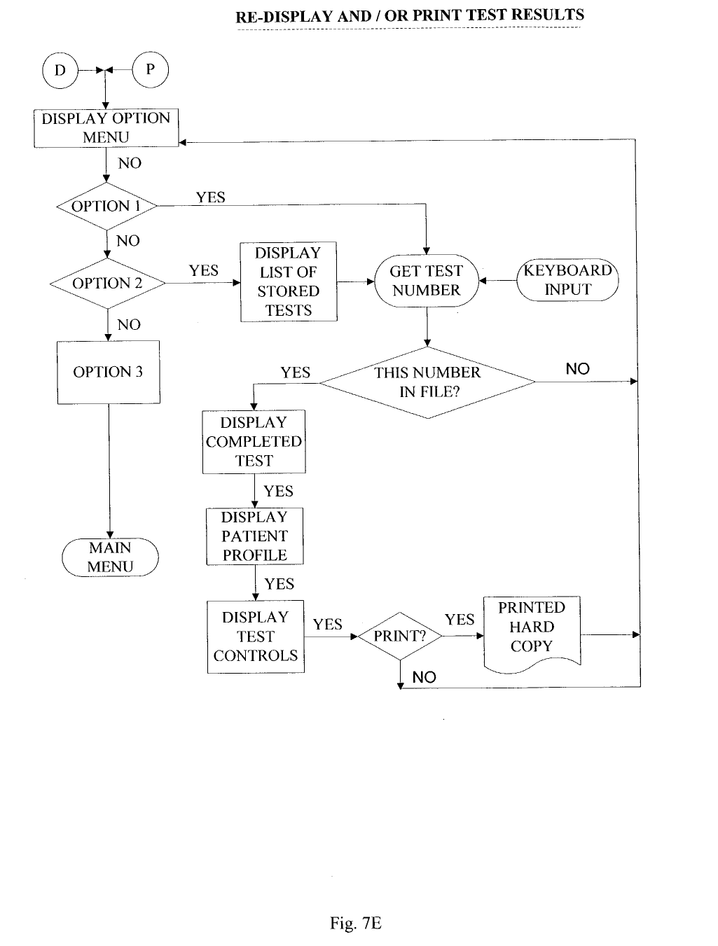

The test software program is presented in FIG. 7 to 7E. The main menu of FIG. 7 is a proposed front end to the software program. This front end permits six major options to be selected: options 1, 2, 3, 4, 5 and 6. These are the same six options displayed in FIG. 7A. FIG. 7B develops option 1. FIG. 7C develops option 2. FIG. 7D develops options 3 & 4. FIG. 7E develops options 5 & 6. These are the routines that either the testing physician or the test taker might need to access. These options can be selected with an input device, such as keyboard 38, button box 40 or some other input device. As depicted in FIG. 7A each option is selected and entered using keyboard input.

These subroutines are representative of a preferred embodiment. However, the subroutines can be modified if desired. For example, the test program presented in FIG. 7A can be started automatically when the system is turned on or it can be selected and turned on using an input device, such as a keyboard, mouse, or keypad. Once the program is started, the program identification can be automatically displayed, if desired by the programmer with a pause for keyboard input, or it can start with a display of test type options, if it is desired to program the system with more than one option.

In FIG. 7A the program is started. At the start the program ID is displayed, after which the user can strike any key on a keyboard 38 or button box 40 to automatically display the test types available. Subsequently, a patient number is displayed, if the ID is known, then other patient information is also displayed. If, however, the patient information is not known, then Option 1 is selected.

Option 1 utilizes the routine presented in FIG. 7B. If the patient number is known, it is input via a keyboard. If it is not known, then an effort is made to upload the patient list from a patient file. If the patient is on the list, then the user number can be input and the system will upload further information form the server and then send the information to temporary storage to be used in the test. Otherwise, the patient information will have to be manually entered, a file created, and the information sent to temporary storage for use in the test. The subroutine provided in FIG. 7B is useful. It helps to identify the test taker and integrates useful information. The person's personal history, visual acuity, pupil size and the like permit a meaning basis for insuring that the test results match up with the right person and permit further use of the information on behalf of the patient.

Once the patient profile is obtained, the test can be run. The procedure is presented in the functional flow chart presented as FIG. 7C. The display target screen is displayed first, followed, by the test controls and patient controls. The start command is entered and then a target is randomly placed in the test area: the macular or the paramacular area depending upon the way the program was set up. The first target is displayed for a non-specific amount of time, generally within a range of 1 to 5 seconds, although a range of 2 to 4 seconds is preferred. The non-specific amount of time is generally varied to avoid having the patient feel that the timing of the test can be anticipated. Then the target is erased again for a varied time period for the same reason. Generally the time can vary from 1 to 5 seconds, but preferably will vary from 2 to 4 seconds. Then another target is presented, again on a random basis with respect to positioning.

If the target is seen, the patient presses a button that sends a signal to an EIA compliant device that send the information to the computers EIA compliant interface. The signal is transferred operating system and then to the test software which then proceeds to upload it to the patient file system.

The target can be varied in size with a larger target being utilized for screening tests and a smaller target being displayed for the complete diagnostic test. Ideally, the target size and the number targets are correlated to completely test the entire area of the macular region if the macular is being tested and the entire area of the paramacular region if the paramacular is being tested.

A round or nearly round target is preferred. However, other geometrical shapes such as squares, triangles, tear shapes, and the like can be employed. The purpose of the test is to determine whether or not the patient saw the target.

In FIG. 7D, routines are presented for changing background colors and for changing target controls. The routines are similar. The target screen is first displayed, and then display options are presented. A choice of options is entered using a keyboard. For either the test colors menu or the test type, select the option that is desired and enter yes to select that option. A no response exits to the main menu.

Using the functional flow chart present in FIG. 7D, the test results can be printed onto paper or displayed on a screen.

An image of a test screen for the evaluating macular degeneration is depicted in FIG. 3. The invention is directed to a method and apparatus for determining the presence of macular degeneration. The method utilizes specialized software has been developed for this purpose and a procedure.

1). A method for detecting vision loss due to disorders of the macular portion of the eye comprising the steps:
k) providing a color video display monitor having a display of three concentric areas, an innermost area corresponding to macular vision, an immediately surrounding second concentric area corresponding to paramacular vision and an outer third concentric area corresponding to extra-macular vision; a fixation point at the center of the concentric areas and cross-hairs as an aid in finding the fixation point;
l) positioning a person at distance from the display suitable for his macular, paramacular and extra-macular vision to correspond to the three concentric test areas,
m) selecting the macular area for testing the patient;
n) recording to a patient demographic file the identity of the person and the eye selected for testing;
o) painting a target in the desired concentric test area in a manner that appears random to the person for a period of time suitable for testing for the ability of the person to see that target,
p) providing a response mechanism to the person to indicate that he is seeing the target, wherein the response mechanism is suitably responsive to permit a timely response;
q) detecting the response of the person to the target;
r) recording to the patient demographic file any lack of response;
s) repeating steps “e” through “g” by redisplaying the target randomly both temporally and spatially in the desired test area until the complete area has been tested, and then
t) recalling the patient demographic file containing the targets not seen, the identity of the person and the eye tested and presenting it on the display or other medium.

2). The method according to claim 1, wherein the patient demographic file is sent to a printer and printed on paper to provide an image of the area of vision loss.

3). The method according to claim 1 wherein each concentric area is defined by a mathematically regular polygon or circle.

4). The method according to claim 3, wherein the mathematically regular polygon is a square.

5). The method according to claim 1 wherein the fixation point is at the center of the display.

6). The method according to claim 1 further including a changeable background color.

7). The method according to claim 1, further including a changeable target color.

8). The method according to claim 1 wherein the patient is positioned approximately 14 inches from the display.

9). The method according to claim 1 wherein the size of the targets can be adjusted.

10). The method of claim 9 wherein a target can vary from ¼ inch to ⅛ inch or equivalent based on a 14 inch viewing distance.

11). The method according to claim 1 wherein the target is a filled-in geometric figure such as a circle, square or other mathematically regular geometric shape.

12). The method according to claim 1 wherein the target appears to the person taking the test as occurring randomly, both temporally and spatially.

13). The method according to claim 1, wherein every target not responded to is displayed on the color video display when the test is completed.

14). The method according to claim 1, wherein a positioning device is used to position the person taking the test.

15). The method according to claim 14, wherein the positioning device is a cone shaped apparatus having an opening that aligns with the fixation point on the color video display and through which the person views the color video display screen.

16). The method according to claim 14, wherein the positioning device is a combination chin/head rest that aligns the eye of the person being tested with the fixation point.
